(19) United States
(12) Patent Application Publication
(10) Pub. No.: US 2013/0012596 A1
(43) Pub. Date: Jan. 10, 2013

(54) POLYMERIZATION OF ALKYL(BETA-N-ALKYLAMINOPROPIONATES) FORMING UNIQUE POLYAMIDE COMPOSITIONS

(76) Inventor: Robert B. Login, St. Marys, GA (US)

(21) Appl. No.: 13/348,055

(22) Filed: Jan. 11, 2012

Related U.S. Application Data
(60) Provisional application No. 61/519,484, filed on May 23, 2011.

(51) Int. Cl. C08G 69/08; A61K 47/34

(52) U.S. Cl. 514/772.3; 528/310; 528/317

(57) ABSTRACT

A process for preparing poly N-substituted beta-alanines with the steps of:

combining in a Michael reaction approximately 1.0 to 1.2 moles of methyl acrylate with approximately one mole of one or more primary amines to produce an alkyl(beta-n-alkylaminopropionate); and

heating the alkyl(beta-n-alkylaminopropionate) to a reaction temperature between approximately 100 and 200 degrees C. and simultaneously evacuating the alkyl(beta-n-alkylaminopropionate) to remove volatiles, thereby forming a poly N-substituted beta-alanine.

This application claims the benefit of U.S. Provisional Patent Application Ser. No. 61/519,484, filed May 23, 2011.

### TECHNICAL FIELD AND BACKGROUND

The present invention relates to polyamides useful as, for example, pharmaceutical drug delivery vehicles, and/or surfactants, encapsulants, protective colloids, thickeners, and so forth, and more particularly to polyamides of N-substituted poly(beta-alanines) with said variety of useful properties.

Degradable polymers are employed for a variety of uses such as the delivery of poorly water soluble (and/or controlled release) of pharmaceutical actives (Polymeric Drug Delivery 11, S. Svensoned; ACS Symposium Series 924, 2006). Such polymers are well known in the medical arts and are usually polyesters with backbone linkages that can hydrolytically degrade. Their hydrolysis byproducts are non-toxic and readily eliminated from the human or animal body. Such polymers serve an extremely important function in medical technology because many drugs, especially those used to treat cancer, are very difficult to deliver to target organs. The usual reason is that they are very insoluble in water, or destroyed or eliminated before they reach the target. The present strategy is to suspend, dissolve, or attach (pro-drug) said drugs to degradable polymers. The polymers containing said drugs can be implanted or injected for example into a cancerous tumor before or after surgery to deliver the cancer drug by erosion, hydrolysis or swelling (osmotic) of the polymer matrix. The drug in ultra-fine amorphous, microcrystalline, or attached (pro-drug) form avoids the solubility and others problems and can then attack the cancer. Many lives have been saved by the use of these ingenious degradable polymers.

Several problems have emerged that compromise the utility of said polymers. Chief amongst them is the cost of the monomers, and the cost to manufacture said polymers. Another problem is the time it takes for said polymers to be eliminated from the body because hydrolysis is rather slow as the polymers can be hydrophobic. To solve this problem, poly(ortho-esters) and poly(anhydrides) were developed. They are more readily hydrolyzed and of significant current interest as drug delivery degradable polymers. However, they are also very expensive and difficult to reproducibly synthesize. Many other polymer types, when conjugated with cancer drugs containing platinum or iron molecules, have been evaluated with various levels of success (Synthetic Polymers as Drug-Delivery Vehicles in Medicine; by E. W. Neuse; Pub Med PMCID:PMC238671; 2008). In order for such polymers to be accepted by the medical community, they must be efficacious, non-toxic and readily removed from the body by excretion. Many such polymers have failed on one or more of the requirements. Therefore, a need exists for new polymer candidates. The present embodiment of N-substituted poly(beta-alanines) definitely meets the economic issues and because of their similarity to peptides, should pass the other hurdles.

To address problems in the art, polymers formed by the polymerization of selected alkyl (beta-n-alkylaminopropionates) (referred to herein as ABNAPs) are prepared by the reaction of primary amines with alkyl acrylates on essentially a one to one mole basis to form Michael adducts. Such ABNAPs, when heated to proper reaction temperatures under conditions discussed below, form by ammonolysis unique polyamides. Mixtures of primary amines can be employed to tailor make such polymers for a wide variety of applications one of which is for the efficacious delivery of drugs to the body.

Several advantages of one or more aspects of the invention process and product by process are as follows: for example, ethanolamine (EA) and methyl acrylate (MA) are readily available very inexpensive raw materials. Their Michael reaction is facile and complete. Conversion to polymeric N-substituted beta-alanines proceeds at reasonable rates at modest temperatures in very high yield. The pendant alcohol groups can continue to react with terminal carboxylic esters to form crosslinks resulting in polymers of very high molecular weight. At a convenient stage of the polymerization, pharmaceutical actives can be formulated, in several ways, with the polymer. Although gelation of the reaction mixture is possible, it was unexpectedly difficult to achieve in the usual polymer synthesis equipment but is possible in suitable equipment after initial polymerization. Tough, flexible, tack-free plastics are produced that will slowly and completely dissolve in aqueous media releasing said actives. Other advantages of one or more aspects will be apparent from a consideration of the drawings and ensuing description.

Prior art reveals that certain Michael derivatives, such as beta-alanine prepared from acrylic acid (or its lower alkyl esters) and ammonia, can be polymerized to nylon 3. In addition, similar polymers can be prepared from acrylamide catalyzed by strong bases (J. Macromol. Sci.-Revs. Macromol. Chem., C6(2), 237-283 (1972) Kennedy & Otsu, eds.). The esters of beta-alanine can be considered the “simplest ABNAP” and its polymerization to nylon 3 (a polyamide) is well documented. However, it can also be considered as one of the ABNAPs of this disclosure in that it might be used in combination with the other ABNAPs, as one of the many types of ABNAPs that can be employed in co- or terpolymers. The prior art however failed to see the possibilities of the polymerization of ABNAPs in various combinations and the novelty and utility of the subsequent polymers. Hydroxyalkylamines, especially ethanolamine's ABNAP, not only polymerize to form polyamides but also cross-link to high molecular weight polymers, even to gels, which are remarkably water soluble.

Although Ogata and Asahara (Bull. Chem. Society of Japan, V. 39, pp 1486-1490 (1966) comment on the polymerization of the ABNAP of ethanolamine, they did not disclose the various embodiments and processing steps disclosed herein.

The components of the ABNAPs and subsequent polyamides are the multitude of primary amines and poly primary amines and the low molecular weight esters of acrylic acid (and possibly methacrylic acid, however, because the alpha methyl group of the methacrylates sterically hinders said polymerization, it will enter said polymerization under more extreme conditions and to a lesser extent), for example, the methyl and ethyl esters. Such amines can also contain other functional groups or structures as long as they do not interfere with the polymerization of the ABNAPs prepared from them.

There is an extensive literature concerning the Michael reaction of various amines with acrylates and methacrylates dating back to the 1930's (U.S. Pat. No. 2,017,537). Many examples show how the reaction of polyamines can produce polyamides (U.S. Pat. Nos. 2,146,210; 3,305,493, and JOC, Dickerman and Simon, March 1957, p. 259). Such amine containing polyamides can be used to react for example with epichlorohydrin forming cationic paper additives (U.S. Pat. No. 6,667,384 and the ref. cited therein). Tsou U.S. Pat. No. 3,145,195 illustrates how a polyamine reacts with half an equivalent of methyl acrylate versus one equivalent of a diamine in water to produce a water soluble polyamide. Similarly, U.S. Pat. No. 3,305,493 shows how one mole of acrylate and one mole of diamine form a Michael adducts that can also react further to form a polyamide. The key difference between my embodiment and these prior art examples is that they do not suggest nor do they show or claim the polymerization of the ABNAPs themselves. What they show is the further reaction of the ABNAP intermediates with additional poly primary amines. The ABNAPs are secondary amines and require heat and vacuum to form polyamides at a reasonable rate. Adding poly primary amines to ABNAP like intermediates causes the reaction of the ABNAP's ester groups with these primary amines because primary amines are much more reactive than the secondary amines in the ABNAPs themselves.

McDonald (US2005/0043506 and U.S. Pat. No. 6,495,657 amongst others) takes an alkyl amine such as tetradecylamine and reacts it with two moles of maleic anhydride to form an intermediate bis(anhydride) that can then be reacted with a polyamine that reacts with the anhydride groups to form a polyamide. This is very similar to Bonvicini (U.S. Pat. Nos. 3,668,278 and 3,773,739) wherein a primary amine is reacted with two moles of acrylate ester and then chain extended with a polyamine to form a tertiary amine containing polyamide similar to McDonald. In each case the bis-Michael adduct of a primary amine and two moles of Michael acceptors produces a tertiary amino bis(anhydride or ester). The resulting tertiary amine bis(anhydride or ester) will not form amides by themselves; therefore, the need for the second polyamine to react with the ester or anhydride to form the polyamide.

Fatty primary amines have been condensed with acrylate esters followed by subsequent ester hydrolysis to afford surface active zwitterionic products. U.S. Pat. No. 2,816,911 illustrates how to avoid residual unreacted fatty amine, an irritant, by using a small excess of the acrylate ester and not heating the initial Michael adduct to higher temperatures to avoid amide formation (such amide formation is central to my embodiment). U.S. Pat. No. 5,922,909 reviews this zwitterionic chemistry and it's references are incorporated in this application.

Recently the Michael reaction has been used to prepare dendrimers (Tomalia et. al., U.S. Pat. Nos. 4,558,120 and 4,568,737) and amino-ester polymers (U.S. Pat. No. 6,998,115).

Reaction with ammonia affords beta-alanine which can be polymerized to nylon 3 and many patents show how to improve this strong base catalyzed process (U.S. Pat. Nos. 4,283,524; 4,459,394 and etc.). Coffey (U.S. Pat. No. 4,609,722) shows how copolymers of beta-alanine precursors and for example 6-aminocaproic acid can polymerize to form nylon-3/6 copolymer. N-substituted polyamides of this embodiment would be counter-intuitive to the goal of preparing high molecular weight nylons because the N-substituant would sterically interfere with the polymerization.

Hollingworth (WO 00/17254; U.S. Pat. Nos. 6,153,724; 6,541,601) and Huang et. al. (U.S. Pat. No. 6,399,714) and McDonald et. al. (U.S. Pat. No. 6,797,743) show how polyamides can be prepared from the Michael reaction of alpha, beta-unsaturated lactones, particularly 2(5H)-furanone and amines. They claim a variety of applications. This lactone is available but is very expensive (Alpha Aesar, a supplier, lists said lactone for $658 per 25 grams). The various embodiments as disclosed herein are very superior as far as cost and availability of starting compounds is concerned. These patents do not teach how to prepare or use ABNAPs.

### SUMMARY

A process for preparing poly N-substituted beta-alanines comprising the steps of:
(a) combining in a Michael reaction approximately 1.0 to 1.2 moles of methyl acrylate with approximately one mole of one or more primary amines to produce an alkyl(beta-n-alkylaminopropionate);
(b) heating said alkyl(beta-n-alkylaminopropionate) to a reaction temperature between approximately 100 and 200 degrees C. and simultaneously evacuating said alkyl(beta-n-alkylaminopropionate) to remove volatiles, thereby forming a poly N-substituted beta-alanine.

The process of above, wherein an alkoxide catalyst is added between step (a) and step (b) in an effective amount of approximately 0.1-1.0%.

The process of above, wherein said alkoxide catalyst is chosen from the group of alkoxide catalysts consisting of sodium alkoxide, potassium alkoxide and titanium alkoxide.

The process of the first paragraph above, wherein said one or more primary amines is ethanolamine.

The process of the first paragraph above, further comprising the step of transferring the poly N-substituted beta-alanine from a first reactor to a second reactor while said poly N-substituted beta-alanine is in liquid form, then heating and evacuating said poly N-substituted beta-alanine to further polymerize said poly N-substituted beta-alanine.

The process of the first paragraph above, further comprising the step of adding pharmaceutical drugs to said poly N-substituted beta-alanine while it is in liquid form.

The process of the fifth paragraph above, further comprising the step of adding pharmaceutical drugs to said poly N-substituted beta-alanine while it is in liquid form.

The process of the first paragraph above, wherein said one or more primary amines is an alkanolamine.

The process of the first paragraph above, wherein said one or more primary amines is a poly-primary amine.

The process of the first paragraph above, wherein step (a) further comprises adding a carboxylic acid, its ester or a di-carboxylic acid.

The process of the paragraph above, wherein said carboxylic acid, its ester or a di-carboxylic acid is added at a maximum ratio of one mole to three moles of said alkyl(beta-n-alkylaminopropionate).

A polyamide composition synthesized by the steps of:
(a) combining in a Michael reaction approximately 1.0 to 1.2 moles of methyl acrylate with approximately one mole of one or more primary amines to produce an alkyl(beta-n-alkylaminopropionate);
(b) heating said alkyl(beta-n-alkylaminopropionate) to a reaction temperature between approximately 100 and 200 degrees C. and simultaneously evacuating said alkyl(beta-n-alkylaminopropionate) to remove volatiles, thereby forming a poly N-substituted beta-alanine;
whereby said polyamide is a N-substituted poly(beta-alanine) with a secondary amine and a carboxylic ester or acid at terminals of said polymer corresponding to the following structure:

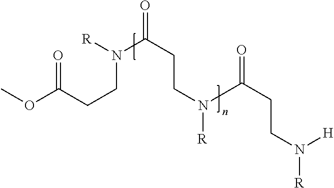

wherein n is between about 3 and 20, wherein each R is between 1 and 50 carbon atoms alone and is optionally substituted with heteratoms, oxygen, nitrogen, sulfur, or phosphorus and combinations thereof and R is either R or a duplicate polymer chain to that shown.

The composition of the paragraph above, wherein the primary amine is selected from the amine formula H₂NCH₂CH(R)OH, wherein R is H or CH₃ radicals.

The composition of the 12^{th} paragraph above, wherein the primary amine is a mixture of said alkanolamine and RNH₂, wherein R is between 1 and 50 carbon atoms alone and is optionally substituted.

The composition of the paragraph above, wherein before said polymerization synthesis step, RCO₂R′ wherein R contains between 1 and 50 carbon atoms alone and is said optionally substituted and R′ is lower alkyl C1-C4 or hydrogen and this is added in the ratio of 3 to 25 moles of said alkanolamine to one mole of RCO₂R′.

The composition of the 12^{th} paragraph above, wherein the mixture of said amines includes a fraction between 1 to 60% of a poly primary amine of structure H₂NRNH₂, wherein R is a diradical of 2 to 24 carbon atoms.

The composition of the 12^{th} paragraph above, wherein the polyamide in liquid form is transferred to a heated vacuum oven or desiccator in order to further polymerize and/or cross-link and cure.

### BRIEF DESCRIPTION OF THE DRAWINGS

FIG. 1 illustrates a starting monomer mix from EA & MA.

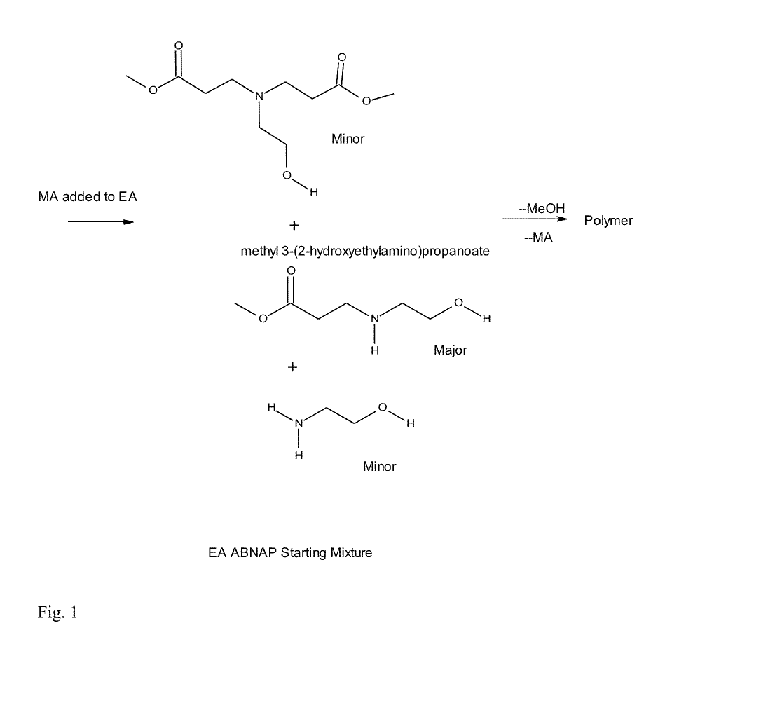

FIG. 2 illustrates a general polymer structure.

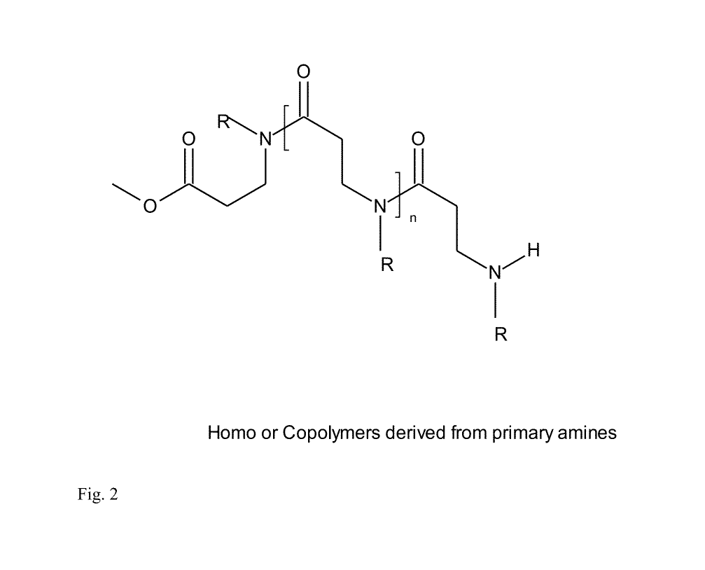

FIG. 3 illustrates a cross-linked general polymer structure.

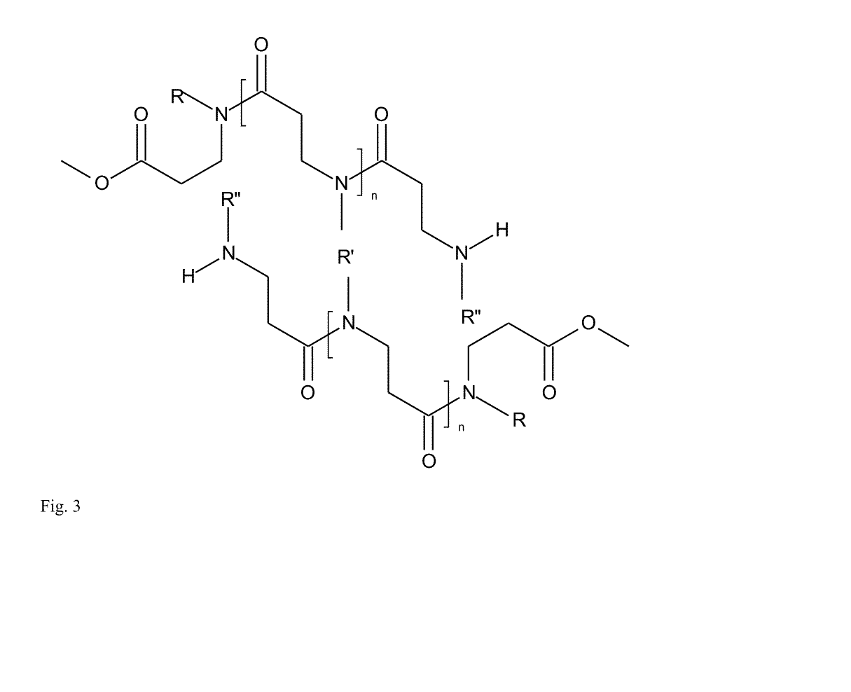

FIG. 4 illustrates a fatty acid terminated, EA based, ABNAP polymer.

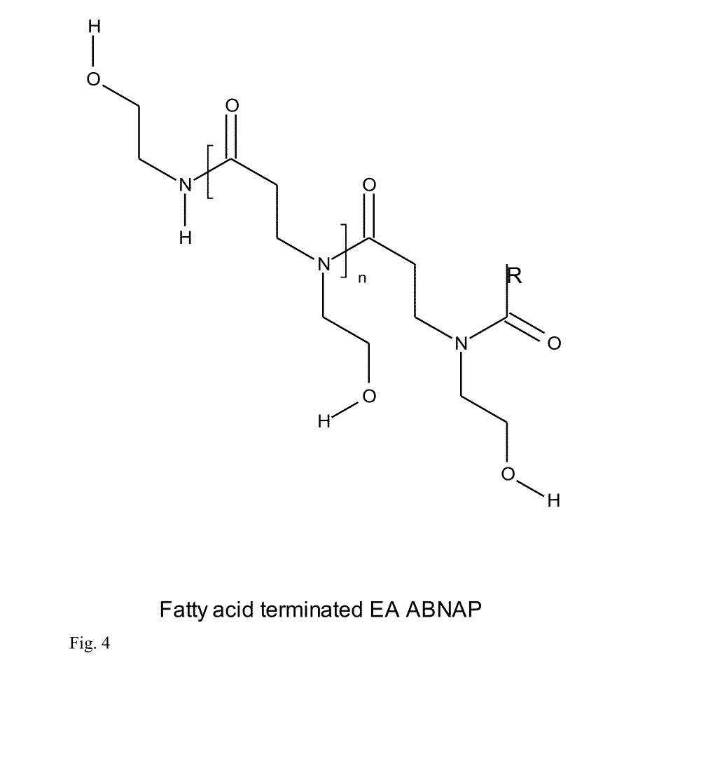

FIG. 5 is an IR spectrum of an EA based ABNAP polymer.

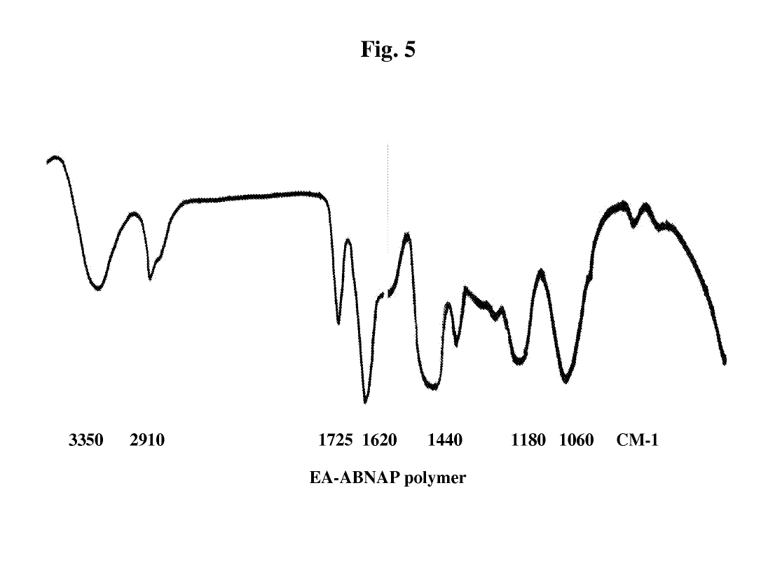

FIG. 6 illustrates two EA ABNAP polymer samples precipitated out of water with IPA showing reduced ester and carboxylate buried under broad amide absorptions.

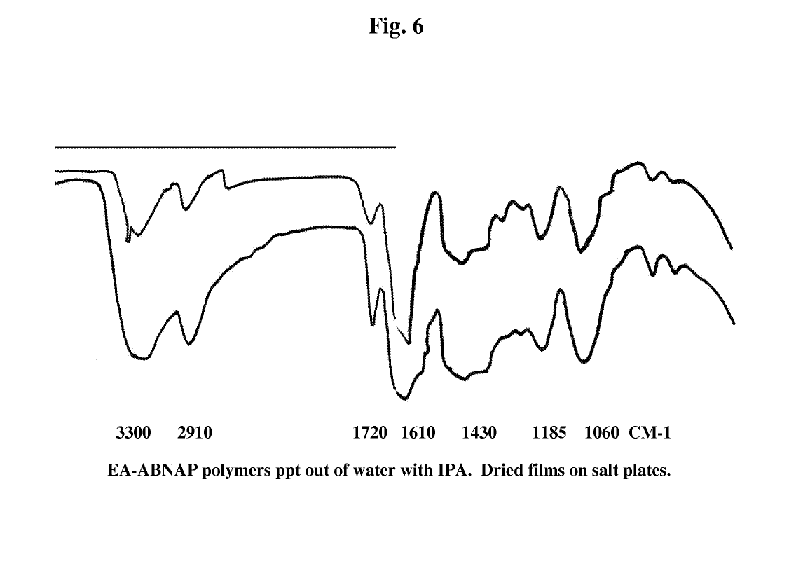

FIG. 7 illustrates lauric acid condensed with EA ABNAPs in 6:1 and 12:1 (ABNAP to LA) ratios.

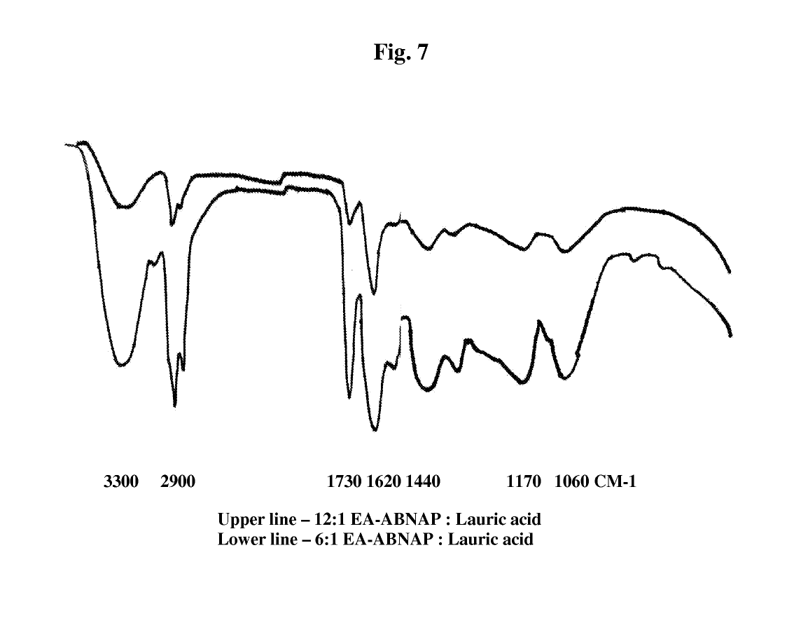

### DETAILED DESCRIPTION

Polymers formed by the polymerization of selected alkyl (beta-n-alkylaminopropionates) (referred to herein as ABNAPs) are prepared by the reaction of primary amines with alkyl acrylates on essentially a one to one mole up to a 1 to 1.2 mole basis to form Michael adducts. Such ABNAPs, when heated to proper reaction temperatures (approximately 100-200 degrees C.) with vacuum removal of volatiles, as discussed in more detail below, form by ammonolysis unique polyamides. Mixtures of primary amines can be employed to tailor make such polymers for a wide variety of applications one of which is for the efficacious delivery of drugs to the body. In one embodiment, methyl acrylate is added drop-wise to the reaction mixture of ethanolamine (and optionally other primary amines or mixtures thereof) maintained at or below about 50 C (in order to reduce premature amide formation, a chain terminator) to prepare ABNAPs, singly or as mixtures. This affords the desired ABNAPs in very high yield. Such ABNAPs can be polymerized to various N-substituted nylon 3 derivatives directly without further purification. At the high temperatures (130-190 C or so) employed to do this ammonolysis reaction, a slight equilibrium apparently exists between the ABNAP, bis-adducts, free amine, and free acrylate ester. In fact, the methanol containing distillate byproduct generally contains some acrylate ester (1-5%). Distilling out acrylate ester along with methanol has an adverse effect on generating higher molecular weight polyamides. This is avoided by using a vigreux column between the reaction flask and the condenser, adding to begin with a small excess of methyl acrylate and refluxing the ABNAP mixture for a time before removing methanol.

Bis-adducts can still form because of the excess MA employed and remained in the final product. The amount of carboxylic acid titer formed by hydrolysis would not equal the amine titer as determined by titration. Even the EA-ABNAP which shows equal titers must still contain some bis-aduct containing polyamides. Other ABNAPs can produce polymers that also retain some bis-adducts.

The methanol that initially forms contains some acrylate ester but it can be refluxed for a time affording the acrylate ester more time to react. However, the temperature to achieve reflux is gradually reduced as methanol is generated in increasing amounts, which by refluxing, cools the reaction temperature to below where ammonolysis can readily occur. When this occurs, low boiling point volatiles are carefully removed through the vigreux column by switching from reflux to distillation. By doing so, this allows the temperature to increase back up until the optimum reaction temperature is reached (130-190 or so). At this point the reaction must not be treated in such a way as to remove ABNAPs which are high boiling compounds that could be removed if too high a vacuum and temperature is employed for example near the end of the polymerization. Not taking these precautions (initial refluxing followed by distillation through a vigreux column) causes an imbalance with the removal of acrylate, for example, generating free amine that can react with the forming polyamide ester end groups terminating the polymerization and if ABNAP is removed, lowering polymer yield. If volatile amines were used to synthesize the ABNAPs, they can also be removed and therefore, care must be exercised by checking the composition of the removed volatiles by GC. Such an analysis will suggest a corrective procedure to rectify the problem such as lower temperatures or longer reflux or combinations.

Catalysts such as sodium methylate may allow the ammonolysis to occur at lower temperatures such as 100-140 C or so. It can also have a positive effect on color, depending on the structure of the ABNAPs employed in the polymerization. With or without the use of a catalyst, once methanol production slows, vacuum can be applied to foster its further removal. Vacuum eventually can be intensified to 25-30″Hg in order to remove the last traces of methanol thus increasing molecular weight and conversion.

The resulting polyamide is usually a viscous liquid that can be cooled to a hard solid thermo-plastic or a very viscous liquid. It can also be added in its molten state to a suitable solvent, for example, water and alcohols where it will dissolve or disperse depending on composition. The resulting polyamide in liquid form can also be placed in a suitable receptacle and then placed in a heated vacuum oven or desiccator. When heated to 100-160 C under 25-30″Hg vacuum for several hours, further polymerization can occur. The reason for doing this is because the regular reactors used for polymerization may not be able to handle mixing a very viscous or cross-linked polyamide. Also upon cooling after such ex-reactor treatment, the polymer is easier to remove and work with.

Pursuing this chemistry with various primary amines led to further insights. N-octylamine's ABNAP afforded a low viscosity product, still a honey like liquid that, depending on molecular weight, solidified upon cooling to a hard wax or very viscous liquid. Its IR was a near duplicate of ethanolamine's ABNAP based polyamide (sans OH absorption) with little ester and major amide adsorption bands. Employing sodium methylate as a catalyst results in lower reaction temperatures, less colored polymers and relatively higher molecular weight but is not absolutely necessary to generating these nylon 3 derivatives but can afford these distinct advantages.

The reaction of benzylamine's ABNAP require a temperature of up to 190 C to generate a methanol-based reflux. This is an example of the benefit of a catalyst such as sodium methoxide to lower the reaction temperature. However, at the end of this reaction at the highest temperature a small amount of higher boiling compound distilled over under vacuum. IR analysis of this compound revealed it to be an ester and not the corresponding acrylamide monomer or other pyrolysis products. Its IR is comparable to the original adduct of the starting amine and methyl acrylate (ABNAP). This indicates that even at 190 C pyrolysis is not taking place. It also illustrates the mistake of removing ABNAP prematurely.

Isopropanolamine's ABNAP, a molecule with less reactive secondary alcohol groups should be less likely to form chain extended polyester linkages. This is the case as this ABNAP formed a much less viscous melt under similar conditions to the reaction of ethanolamine's ABNAP. Upon cooling, the product set up to a very viscous dense liquid. This result indicates that ethanolamine's ABNAP based polyamides exhibit polyester linkages that account for its higher melt viscosity. Sodium methylate catalyzes the ethanolamine based ABNAP polymerization affording even higher molecular weight polymers. In fact, the ethanolamine based ABNAP polymers can be gelled if conditions are vigorous enough such as employing very high vacuum and temperature, but the reaction is slow and if desired, gelation is easily avoided. This result is an unexpected discovery as those chemists of ordinary skill in this art would expect such a monomer to be easily gelled when polymerized with sodium methylate because of the plethora of hydroxyl and ester groups.

Every polymer so far produced exhibits a similar IR spectrum as concerns the ester/amide bands. A small ester carbonyl band but major broad amide carbonyl bands are apparent.

The polyamides produced by said process are relatively low molecular weight polymers or oligomers. Those prepared from the EA-ABNAP when dissolved in water because of ester cross-link hydrolysis, are also oligomeric. Their relative molecular weight is determined by titration of the amine end groups as determined in aqueous or alcoholic solutions. However, suitable catalysts such as the strong bases like sodium methylate can be employed to increase molecular weight. Even though said polyamides are low molecular weight (500-4000 amu or so) polymers (oligomers), they are very valuable products. For example copolymers of ethanolamine's ABNAP and fatty amines such as octylamine's ABNAP or dodecylamine's ABNAP in various proportions can afford upon neutralization of the residual amine groups, clear aqueous pH7 solutions that can be rendered opaque at either slightly higher pH's or by heating their solutions to somewhat higher temperatures similar to the cloud points observed with many surfactants. These properties have been referred to as being associated with “smart polymers” (for example, Chapter 6 in Topics in Tissue Engineering, Vol. 3, 2007 and U.S. Pat. Nos. 7,157,603; 7,625,764 and others). Dodecylamine/ethanolamine's ABNAP (20/80%) copolymer is very surface-active generating relatively stable foams from dilute aqueous solutions. Surface tension of 28 dynes/cm are found at its CMC (a 0.007% solution). More concentrated aqueous solutions are viscous presumably because of associative long alkyl chain interactions. The copolymers containing 40 mole % of dodecylamine's ABNAP and 60 mole % ethanolamine's ABNAP are (upon neutralization to pH 7) viscous water-soluble lubricants that feel very dry and smooth on the skin. The ester terminus of these polymers depending on pH is not hydrolytically stable and will form the carboxylic acid or salt in aqueous solution resulting for example in increased solubility. Such ester-hydrolyzed polymers can be considered peptide analogs.

The ethanolamine ABNAP based polymers are clearly cross-linked by ester linkages. Post treatment in a heated vacuum container affords flexible rubbery non-melting polymers that are clearly cross-linked high molecular weight materials. They however will readily dissolve in water to form solutions similar to those obtained when said polymers are in the thermoplastic melt-able state. Careful titration of both using an auto-titrator with graphical print out shows similar titers for both carboxylic and amine pKa's and similar molecular weights calculated from the titer assuming they are end-groups of about 400-700 amu or 4-6 repeating monomer units. This is very important because the size of the said hydrolyzed polyester-polyamide will determine its ease of removal from a biological system after drug release. Said polyester-polyamides start as high molecular weight plastics but readily hydrolyze to oligomers that can be excreted from the human body.

The process, limited only by the availability and reactivity of primary amines, can be used to prepare homo, co- and terpolymers. Thus a wide variety of amino and acid terminated peptide like polymers can be prepared that as previously mentioned are of interest in various areas of biochemical and medical technology. Other uses are possible for example, they can function as surfactants, protective colloids, foam stabilizers, detergent auxiliaries, solubilizers for actives in ointments and deliver active ingredients in cosmetic, personal care and pharmaceutical formulations. These polymers contain a secondary amine at one end of the polymer and a carboxylate ester at the other end both of which can be attachment sites for attaching pro-drugs for the delivery of pharmaceutically active target molecules. The terminal amine group can also be employed to catalyze epoxy curing or be incorporated in urethane systems. Said polymers could also function as tackifiers in a variety of adhesives. The ability to tailor the polymers by the selection and mixture of primary amines enhances the utility of this embodiment. Those skilled in the art can conceive of numerous other uses. The above suggested uses for said embodiment are given as examples and are not meant to be limiting.

The preparation of said polymers is an example of “green” chemistry because no solvents are employed in their manufacture and minimal byproducts are formed. The reaction of a primary amine or mixture of primary amines with, for example, methyl acrylate can be done neat with no solvent. The reaction is exothermic and affords nearly 100% products that can be further converted to polymer by removing methanol as it forms under reaction conditions. The fact that the polymerization of those polymers prepared with ethanolamine do not form gels even with sodium methylate unless really pushed (higher temperatures and stronger vacuum) indicates that the amide linkage is the thermodynamic end point of this chemistry and if esters form from the pendant alcohols they usually end up as mostly amide linkages. Only under vigorous reaction conditions of high vacuum and temperature, has gelation been observed with ethanolamine's ABNAP. In fact, when the reaction product at a stir-able viscosity is discharged into a polyethylene dish as a thin film and placed in a vacuum oven or dessicator at 29-30″Hg and 140 C for up to two hours, a flexible rubbery polymer of low color can result. This polymer would not melt when heated, formed swollen gels in DMF, NMP, and DMSO and would not dissolve in these powerful solvents. This indicates extensive cross-linking. When water is added to this heavily cross-linked polymer, it swelled and dissolved after several hours at room temperature. The fact that useful un-gelled polymers can be obtained from the polymerization of ethanolamine's ABNAP is a very important unexpected discovery. Actives such as pharmaceutical drugs can be added to this polymer when liquid or pressed together employing said polymer in crushed particle form and if necessary, the polymer can then be further cross-linked in a vacuum oven or desiccator, resulting in a polymer that can dissolve in aqueous based body fluids and release said drugs in a useful manner.

As previously mentioned, prior art polyesters employed to deliver drugs, depend on the hydrolytic instability of the ester groups to degrade the polymer backbone so that fragments of the polyester can now be in a form to be eliminated from the body. In the present embodiment, the polyester linkages are actually cross-links that build molecular weight but are also hydrolytically labile producing upon hydrolysis, oligomeric peptide analogs that can be easily eliminated from the body.

### Examples

All chemicals used in the examples were purchased from Alpha Aesar or Sigma Aldrich and used as received. Methyl acrylate was 99% pure and contained 15 ppm 4-methoxyphenol stabilizer. GC analysis were performed on a HP5890 equipped with a HP-1 30 m column and a TCD detector. HPLC analysis were performed on a Waters 600E system with a 490E uv detector and a HP-1037A RI detector. Surface tensions were measured on a Fisher Tensiomat du Nouy balance. Infrared spectra were obtained on a Perkin Elmer model 1310. Auto titrations were performed on a homemade system consisting of an accurate Waters 510 pump and a pH meter whose millivolt output was recorded on a Perkin Elmer strip chart recorder.

The following is an example of a general method for preparing said polyamides: To a multi-neck round bottom flask equipped with a mechanical stirrer, dropping funnel, 300 mm vigreux column, condenser, thermometer, nitrogen sweep and heating mantle is added one mole of primary amine or mixture of primary amines. A means of cooling is made available. To this stirred mixture methyl acrylate (MA) 1.1 moles or so is added drop-wise at such a rate as to keep the temperature below 50 C, by cooling if necessary. A small amount of about 10-20 mole percent excess of MA is included in the charge. If sodium methylate is employed, it is added after the exotherm resulting from the formation of the ABNAPs is over. Less than 1% of a 25% solution in methanol but usually 0.3% (based on the total weight of the ABNAPs) is adequate. (Note: if sodium methylate is used, it must be neutralized at the end of the polymerization. I used glacial acetic acid but other acids can be used.) After the ABNAP exotherm subsides and usually after two hours, heat is applied raising the temperature up until the mixture refluxes. This varies according to the amine or amines used, but is usually in the 100-190 C range.

As reflux continues, distillate is collected from time to time to measure volatiles by GC looking for minimum acrylate ester, or until the reaction temperature drops, because of methanol's reflux cooling to below where ammonolysis is observed (usually 160 C without catalyst and 120 C or so with sodium methylate). When this occurs, the reflux is stopped and the configuration is changed to distillation. Heat is again applied and distillate is collected at atmospheric and then vacuum is gradually applied until best vacuum is achieved (25-30″Hg). The reaction is continued until methanol production slows significantly. To maintain methanol production, the reaction temperature is slowly increased and even temperatures around or above 190 C are acceptable depending on the nature of the volatiles analyzed by GC and the propensity for undesired color. Very good stirring helps the volatiles to escape the reaction mass especially as the production of methanol slows; however, care is taken not to remove ABNAPs.

The finished reaction mass, usually a viscous honey-like light yellow to orange or reddish brown colored liquid, can be cooled to a lower temperature under a nitrogen sweep, optionally neutralized with acid if sodium methylate was used and poured into a suitable container or added to a solvent under nitrogen, with stirring and cooling.

Should further reaction be desired, then neutralization of the catalyst is not done and the molten polymer is transferred to a suitable container and placed in a vacuum oven or desiccator maintained at reaction temperatures of for example 140 C and best vacuum (25-30″Hg) for several hours. This can result in higher molecular weight or extensive cross-linking depending on the ABNAP(s) employed to begin with.

If block, co- or terpolymers are desired, they can be synthesized by the addition of each previously formed ABNAP at judicious intervals under reaction conditions.

The above procedure is not to be construed as limiting, as the number ABNAP structures and variations of polymer synthesis are too numerous to detail in this description but should be apparent to those of ordinary skill in this art. The chemistry of the ABNAPs is very flexible and varied.

For example, the primary amines used to prepare an ABNAP can contain other functional groups as long as they do not hinder polymerization. Primary amines can contain a variety of alkyl, alkylene, aromatic, heterocyclic groups of all types in various combinations. They could contain phosphorus functionalities or various silane or other varieties of silicone containing functionality or contain halogens, transition metal complexes, oxygen, sulfur, and so forth. This would be apparent to those skilled in the art and are too numerous to go into detail here, but the above are examples signally.

The following is an example for an EA-ABNAP homopolymer: Using the general method, 400 g (4.65 moles) of MA was added to 244 g (4 moles) EA followed by 2 g 25% NaOMe in MeOH. Reflux started at 105 C followed by distillation after 40 minutes. Maximum temperature was 135 C at 25″Hg. Product was discharged when stirring became difficult because of viscosity. HOAC 0.56 g were added before discharge. Collected 112.5 g volatiles vs. 128 for 4 moles of MeOH. GC analysis indicates mostly MeOH with some MA. Apparently most of the excess MA formed bis-adduct was incorporated in the polymer. After cooling at 0° C., the brittle plastic could be broken up into smaller fragments that at RT were hard but somewhat flex-able clear to hazy plastics. Up to 52% aqueous solutions could be prepared that were white opaque and thixotropic. Heating such solutions to 75-85 C cleared them to nearly transparent. Dilute solutions are transparent. Auto-titration shows 1.73 meq/g for both amine and acid.

A repeat preparation with identical charges and similar procedures resulted in a very similar polymer with 1.39 meq/g titer for both amine and acid. This time however no HOAC was used to neutralize the catalyst because it was reasoned that upon dilution in water the desired hydrolysis of ester cross-links would be facilitated.

A repeat preparation with 122 g EA (2 moles) and 177 g MA (2.06 moles) and 1 g 25% NaOMe resulted in a polyamide very similar to those made with a greater excess of MA. However, In this case the initial reflux was extended to react out the MA. GC analysis indicated very little MA if any in the distillate.

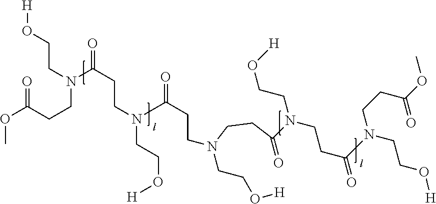

The above structure emphasizes the inclusion of the bis-adduct that contains tertiary amine and the possibility of two ester terminals. With the plethora of hydroxyl groups and NaOMe catalyst, it is no wonder that cross-linking can ensue.

It would be unreasonable to conclude that no bis-adduct would be in the ABNAP mixture no matter the ratio of amine to MA. Adding MA at lower temperatures to the amine is designed to reduce bis-adduct formation but does not eliminate it. The actual structure of the resulting polyamides is therefore a complex mixture of some amide terminated chains, bis-adduct containing and mostly ABNAP containing polymer chains.

Another embodiment utilizes esters or carboxylic acids of for example, fatty acids such as methyl laurate or lauric acid, can be mixed with selected ABNAPs to be incorporated in the subsequent polymer as an end group. This affords new and novel polymeric surfactants that can have very useful properties. Other esters (or acids) such as dimethyl terephthalate or terephthalic acid can also condense by with ABNAPs to form another set of polyamides with unique properties.

In another example, lauric acid is condensed with 3 moles of EA-ABNAP. In the same equipment described in the general procedure, 91.5 g of EA (1.5 moles) was charged and cooled in an ice bath as 142 g of MA was added. The temperature during the addition of MA was kept below 50 C. After the exotherm abated and a half hour, 100 g of lauric acid (0.5 mole) was added all at once. The temperature was slowly increased to 130 C at which point methanol started to distill over. The temperature was slowly raised to 165 C and distillation of methanol slowed. About 55 g of distillate was collected at atmospheric pressure. The mixture was then placed carefully under vacuum until 29+″ hg was achieved at 175 C at which point bubbling ceased and the honey like product was cooled and discharged. A theoretical 263.5 g vs. 263 g of product was obtained. Theoretical volatiles consisted of 48 g MeOH, and 9 g water and 57 g was collected.

In still another example, lauric acid condensed with 6 moles of EA-ABNAP. In the same procedure as above, the following amounts were used: 91.5 g EA, 150 g MA and 50 g lauric acid. 65.5 g volatiles (5 g collected in vacuum trap) vs 73.5 theoretical were collected. Auto-titration of aqueous sample adjusted to pH 2 (also to see if lauric acid would precipitate, but none was observed even after several days), indicated a MW of 1,017 amine (from bis-adduct) and 1525 CO2H, by end group analysis. CMC appears to be about 0.1% at 30 dynes/cm.

In still another example, lauric acid condensed with 12 moles of EA-ABNAP. In the same procedure as above, the following amounts were used: 91.5 g EA, 142 g MA and 25 g lauric acid. 54.3 g vs. 50.3 theoretical and 186 g vs. 195 g theoretical product were collected. Auto-titration as above indicated MW of 2472 CO2H and 1935 amine. CMC appears to be about 0.1% at 28 dynes/cm.

In these three examples, no attempt was made to remove excess MA, therefore bis-adduct containing tertiary amine was incorporated in the polymer mix. Being tertiary, it will not react with the lauric acid; however enough EA-ABNAP reacts to produce a desirable product.

As another example, DMT (Dimethyl terepthate) and 6 moles EA-ABNAP: Using the equipment described in the general procedure, the EA-ABNAP (97.5 g EA and 150.9 g MA) was prepared and to it was added at 40 C, 51.6 g DMT. Heating the mixture dissolved the DMT and at that point, 1 g 25% NaOME in MeOH was added. Reflux started at 105 C and after 15 minutes it was changed to distillation. Highest temperature was 150 C at 25″Hg where volatiles stopped (collected 58 g vs 56 g theoretical). The mixture was discharged into a polyethylene open container which was placed in a heated vacuum desiccator at 120-140 C for one hour at 29+″Hg. The resulting polymer was a very brittle plastic that could be crushed to a coarse powder. It was insoluble in DMF, DMSO, NMP, forming a gel-like material, but slowly soluble in water. This polymer would not melt when heated, turning brown with oxidation.

Poly primary amines, for example, hexamethylenediamine (HMDA), can be used to form poly ABNAPs by themselves or in combination with a wide variety of other ABNAPs. By itself, hexamethylenediamine's bis-ABNAP will eventually gel when polymerized by the general method. However, gelation requires high temperatures and good vacuum; therefore, the intermediate polymer can be discharged from the reactor as a liquid and the gelation can be pursued in a heated vacuum oven or desiccator. As before valuable pharmaceutical actives can be incorporated during the liquid stage and trapped in the subsequent gel. Said gel containing said actives slowly swells over many hours releasing said actives over time.

HMDA can be mixed with other primary amines of all types depending on the desired end-use and reacted with, for example, methyl acrylate to form a mixture of ABNAPs. Subsequent polymerization can result in a new family of polyamides. Said polyamides benefit from the presence of this bis-ABNAP in the mixture because the resulting polyamide can be much more viscous because of branching.

As another example, a homopolymer of HMDA bis-MA adduct: Employing the same equipment and general procedure, 86.09 g of MA (1 mole) was added to 58.1 g of HMDA (0.5 mole). After initial reaction, 1 g of 25% NaOMe in MeOH was added and then the mixture was heated until reflux began at 180 C. As reflux increased, the temperature dropped to 140 C at which point the volatiles were removed by distillation.

When volatile distillation slowed even at 170 C, vacuum was applied. The mixture started to thicken and as stirring became more difficult, the vacuum was relieved and 2.5 g of powdered tylenol was added at 150 C. After a few minutes of mixing, the liquid reaction mass was transferred to a polyethylene open container and placed in a heated vacuum dessicator at 120-140 C at 29+″Hg for 1.5 hrs. The liquid polymer now was converted to a rubber that was easily cut with a scissor, and apiece was placed in water where it swelled but did not dissolve.

A further example, 70/30 mole % Octylamine (OA)/HMDA: Employing the general procedure with 65 g (0.503 moles) OA mixed with 25.6 g (0.22 moles) HMDA to witch was added 89.3 g MA (1.04 moles). No catalyst was employed and the ABNAP mixture was heated to 165 C where reflux began. Eventually 180 C and 29+″Hg vacuum was needed to finish the condensation. Even under these conditions, the color of the polymer was good. About 42.5 g of volatiles (GC mostly MeOH and some MA) vs 33 g MeoH and 8 g excess MA. The OA-ABNAP homopolymer is a mobile liquid while this copolymer is flow able but extremely viscous.

Although it was previously mentioned that other functionality can be part of the primary amines, N,N-dimethylaminopropylamine (DMAPA) is specifically pointed out as an example because it is readily commercially available and confers a pendant tertiary amine functionality to said polyamides. It can be mixed with other amines such as EA, and/or HMDA to afford a variety of polyamides. Such tertiary amine containing polyamides can for example be converted to acid salts, quats, betaines, sultaines and amine oxides. Said derivatives are well known to those of ordinary skill in the art.

Another example: Terpolymer of a 60 mole % (an 80/20 mole % mixture of n-laurylamine/DMAPA) and 40 mole % HMDA: Employing the general procedure with a 90 g (0.49 moles) laurylamine, 12.4 g (0.12 moles) DMAPA, and 47.25 g (0.407 moles) HMDA mixture to which is added 135 g (1.57 moles) MA. No catalyst is used and after the formation of the ABNAP mixture, it is slowly heated to 175 C where reflux begins. Cooling to 160 C resulted in switching to distillation and then after one hour the temperature slowly increased to 180 C. At which point 47.8 g of distillate was collected. Placed under vacuum eventually reaching 29+″Hg at 185 C after another hour. The light colored product was poured out of the reactor to cool to an extremely viscous liquid. Theoretical 209 g of product and 50 g MeOH vs. 198 g recovered and 65 g volatiles (GC mostly MeOH, some MA and complicated high boilers).

The above product was mixed with water and was insoluble even when heated to 70-80 C for one hour. Another sample afforded a viscous aqueous solution when neutralized with acetic acid. Another sample was treated with an equivalent of 35% H₂O₂ at 70-80 C. The two phase mixture became homogeneous and cooled to a white uniform paste.

Ammonia can be added to two moles of MA to afford a secondary amine attached to two ester groups. This is a polymerizable AB2 type monomer.

Example, NH₃ bis-adduct: The bis-adduct monomer was prepared by heating 76 g of a 28% NH₄OH solution, in a separate flask, in order to generate NH₃ gas (which was passed through molecular sieves to remove moisture) which is then bubbled into 237 g of MA in 107 g isopropanol. This exothermic reaction is done with cooling to below 40 C to avoid premature amide formation. After the exotherm abates, isopropanol and unreacted MA are stripped out under vacuum. The rest of the polymerization is identical to the general method. In this case, no catalyst was employed and the maximum temperature was 175 C. The resulting polyamide exhibits an IR spectrum nearly identical to others but shows a larger ester carbonyl absorption.

The following chart summarizes the various polyamides prepared as examples. The amine or amines employed are listed with the understanding that the actual monomer is the ABNAP or ABNAP mixture. “Max C” is maximum temperature reached during polymerization. MW was determined after aqueous dissolution by amine end group titration. Abbreviations were as follows: EA (ethanolamnie); OA (Octylamine); BA (Butylamine); LA (laurylamine); HMDA (Hexamethylenediamine); DMAPA (N,N-dimethylaminopropylamine); DMT (Dimethyl terephpthalate):

TABLE 1

| Amine | Max C | Appearance | MW | Comments (color/cat) |
|---|---|---|---|---|
| EA (1:1.15) | 160 | hard thermoplastic | 488 | water sol/Gardner 6/no cat |
| EA (1:1.16) | 160 | ″ | 1315 | water sol/gardner 3/NaOMe |
| EA (1:1.16) | 135 | ″ | 1,087 | water sol/gardner 2/NaOMe |
| OA (1:1) | 190 | wax | 2,031 | alcohol sol/gardner3/NaOMe |
| BA (1:1.05) | 190 | fluid | 489 | alcohol sol/gardner 3/NaOMe |
| BA/HMDA (1:1.1) | 190 | vy viscous | 542 | alcohol sol/gardner 2/no |
| (85/15 mole %) | | | | |
| BA/HMDA (1:1.1) | 190 | more viscous | 650 | alcohol sol/gardner2/no |
| (70/30 mole %) | | | | |
| EA/LA (1:1.1) | 140 | thermo-plastic | 1311 | visc. aq. soln/gardner 2/yes |
| (80/20 mole %) | | | | |
| EA/LA (1:1.1) | 175 | thermo-plastic | 1875 | visc. aq. soln/gardner 8/yes |
| (80/20 mole %) | | | | |
| OA/DMAPA (1:1.1) | 190 | viscous | 661 | sol. pH < 6/gardner 3/no |
| (80/20 mole %) | | | | |
| [(OA/DMAPA)]/[HDMA] | 185 | more viscous | 529 | sol. pH < 6/gardner 3/no |
| (1:1.1) (80/20)/[70/30]% | | | | |
| [(LA/DMAPA)]/[HDMA] | 185 | more viscous | 673 | sol. pH < 6 viscous/gardner 3/no |
| (1:1.1) (80/20)/[60/40]% | | | | |
| Lauric acid/EA ABNAP | 170 | very viscous | 1600 | no ppt at pH 2/gardner 4/no |
| (1:1.16) (1:6 mole ratio) | | | | |
| HMDA diABNAP (1:1) | 160 | viscous then 140 C. | | gel/gardner 3/NaOME |

It is understood that equivalents and substitutions for certain elements set forth above may be obvious to those of ordinary skill in the art, and therefore the true scope and definition of the invention is to be as set forth in the following claims.

I claim:

1. A process for preparing poly N-substituted beta-alanines comprising the steps of:
(a) combining in a Michael reaction approximately 1.0 to 1.2 moles of methyl acrylate with approximately one mole of one or more primary amines to produce an alkyl(beta-n-alkylaminopropionate);
(b) heating said alkyl(beta-n-alkylaminopropionate) to a reaction temperature between approximately 100 and 200 degrees C. and simultaneously evacuating said alkyl(beta-n-alkylaminopropionate) to remove volatiles, thereby forming a poly N-substituted beta-alanine.

2. The process of claim 1, wherein an alkoxide catalyst is added between step (a) and step (b) in an effective amount of approximately 0.1-1.0%.

3. The process of claim 2, wherein said alkoxide catalyst is chosen from the group of alkoxide catalysts consisting of sodium alkoxide, potassium alkoxide and titanium alkoxide.

4. The process of claim 1, wherein said one or more primary amines is ethanolamine.

5. The process of claim 1, further comprising the step of transferring the poly N-substituted beta-alanine from a first reactor to a second reactor while said poly N-substituted beta-alanine is in liquid form, then heating and evacuating said poly N-substituted beta-alanine to further polymerize said poly N-substituted beta-alanine.

6. The process of claim 1, further comprising the step of adding pharmaceutical drugs to said poly N-substituted beta-alanine while it is in liquid form.

7. The process of claim 5, further comprising the step of adding pharmaceutical drugs to said poly N-substituted beta-alanine while it is in liquid form.

8. The process of claim 1, wherein said one or more primary amines is an alkanolamine.

9. The process of claim 1, wherein said one or more primary amines is a poly-primary amine.

10. The process of claim 1, wherein step (a) further comprises adding a carboxylic acid, or a di-carboxylic acid or esters of said acids.

11. The process of claim 10, wherein said carboxylic acid, or a di-carboxylic acid, or said esters is added at a maximum ratio of one mole to three moles of said alkyl(beta-n-alkylaminopropionate).

12. A polyamide composition synthesized by the steps of:
(a) combining in a Michael reaction approximately 1.0 to 1.2 moles of methyl acrylate with approximately one mole of one or more primary amines to produce an alkyl(beta-n-alkylaminopropionate);
(b) heating said alkyl(beta-n-alkylaminopropionate) to a reaction temperature between approximately 100 and 200 degrees C. and simultaneously evacuating said alkyl(beta-n-alkylaminopropionate) to remove volatiles, thereby forming a poly N-substituted beta-alanine;
whereby said polyamide is a N-substituted poly(beta-alanine) with a secondary amine and a carboxylic ester or acid at terminals of said polymer corresponding to the following structure: 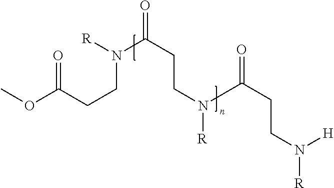
wherein n is between about 3 and 20, wherein each R is between 1 and 50 carbon atoms alone and is optionally substituted with heteratoms, oxygen, nitrogen, sulfur, or phosphorus and combinations thereof and R is either R or a duplicate polymer chain to that shown.

13. The composition of claim 12 wherein the primary amine is selected from the amine formula H₂NCH₂CH(R)OH, wherein R is H or CH₃ radicals.

14. The composition of claim 12 wherein the primary amine is selected from
H₂N[(CH₂)ₓ—CH(R)—O]ₙ—H
where x=1-6; n=1-20; R═H or C1-4 alkyl radical.

15. The composition of claim 12 wherein the primary amine is a mixture of said alkanolamine and RNH₂, wherein R is between 1 and 50 carbon atoms alone and is optionally substituted.

16. The composition of claim 14 wherein before said polymerization synthesis step, RCO₂R′ wherein R contains between 1 and 50 carbon atoms alone and is said optionally substituted and R′ is lower alkyl C1-C4 or hydrogen and this is added in the ratio of 3 to 25 moles of said alkanolamine to one mole of RCO₂R′.

17. The composition of claim 12 wherein the mixture of said amines includes a fraction between 1 to 60% of a poly primary amine of structure H₂NRNH₂, wherein R is a diradical of 2 to 24 carbon atoms.

18. The composition of claim 12 wherein the polyamide in liquid form is transferred to a heated vacuum oven or desiccator in order to further polymerize and/or cross-link and cure.
